(19) United States
(12) Patent Application Publication
(10) Pub. No.: US 2017/0067013 A1
(43) Pub. Date: Mar. 9, 2017

(54) ENHANCEMENT OF RECOMBINANT PROTEIN EXPRESSION WITH COPPER

(71) Applicant: Advantech Bioscience Farmacêutica LTDA, Barueri (BR)

(72) Inventors: Sadettin Seyit OZTURK, Paoli, PA (US); Matthew Veron CAPLE, Chesterfield, MO (US)

(73) Assignee: Advantech Bioscience Farmacêutical LTDA, Barueri (BR)

(21) Appl. No.: 15/119,714

(22) PCT Filed: Mar. 3, 2015
(86) PCT No.: PCT/BR15/00025

§ 371 Date: Aug. 17, 2016

Related U.S. Application Data
(60) Provisional application No. 61/969,215, filed on Mar. 23, 2014.

(51) Int. Cl. C12N 5/00; C12P 21/02

(52) U.S. Cl. CPC C12N 5/0018; C12P 21/02; C12N 2500/20; C12N 2511/00

(57) ABSTRACT

The present invention provides a novel use of copper (cupric ion) for improved cell expression of recombinant proteins, particularly coagulation proteins such as recombinant Factor VIII, B Domain Deleted recombinant Factor VIII, recombinant Factor IX and rFVII or rFVIIa. The use of such cell culture supplement results in higher productivity and robustness of the manufacturing process. This invention results in improvements in cell expression and product stability.

### CROSS-REFERENCE TO RELATED APPLICATIONS

This application is based on and claims priority of 61/969,215 filed 23 Mar. 2014.

### STATEMENT REGARDING FEDERALLY SPONSORED RESEARCH OR DEVELOPMENT

Not applicable

### BACKGROUND

1. Field

Recombinant proteins have been made by cell culturing based on the batch method or perfusion since the 1980s. The present invention provides improved cell expression, particularly in mammalian cells, by the use of copper additives. This invention is applicable to many mammalian cell cultures, such as CHO, BHK and human cell lines, particularly CHO, and to the expression of many recombinant proteins, such as recombinant Factor VIII (rFVIII) B Domain Deleted rFVIII and recombinant Factor VII/Factor VIIa (rFVII/rFVIIa).

2. Related Background Art

Copper is essential for cell growth and survival. Because of copper's essential nutrient value, its chemical role as a catalyst of oxidative stress and its propensity to precipitate, it is critical to understand, monitor and formulate it for use in specific cell culture systems and applications.

Copper is a transition metal that exists, in vitro, in an equilibrium as reduced (cuprous), Cu (I) and oxidized (cupric), Cu (II), copper. In its free form and in some chelates, it can participate actively in redox cycling. It oxidizes a number of important media components, such cysteine and ascorbate, for optimization of the cell culture process.

In vitro, Cu (I) will spontaneously form complexes with reduced cysteine, glutathione and presumably organic sulfhydryls. In addition to forming cupri-cystine complexes, Cu (II) will form complexes with other amino acids through coordination of their alpha-amino nitrogen and carboxyl-oxygen groups. Binding of Cu (II) to histidine is important because this appears to be an intermediate involved in the movement of Cu (II) from albumin to the cell. Before the copper can cross the cell membrane it must be reduced to Cu (I).

Copper can cause the loss of the cysteine and cystine from cell culture media by oxidation and precipitation. In vitro, cysteine is freely soluble and exists almost exclusively as a neutral amino acid. It is unstable and undergoes non-enzymatic autoxidation in the presence of di-molecular oxygen to form cystine. Cupric copper accelerates the autoxidation of cysteine to cystine. Cupric copper can form chelate-precipitates with cystine. The depletion of cysteine from cell culture will stop the synthesis of proteins and glutathione, an important reducing agent. Reduced glutathione can complex with Cu (I) and inhibit its participation in the formation of hydroxyl free radicals. This interaction involves the cysteine sulfur atom. In vivo, Cu (I):glutathione complexes mediate the safe movement of Cu (I) that enters the cytoplasm, probably through the copper transporter 1 pore, to intra-cellular binding proteins such as metallothionein. The formation of Cu (I): glutathione complexes is spontaneous and non-enzymatic, [Dierick, P. J. (1986), In vitro interaction of organic copper (II) compounds with soluble glutathione S-transferases from rat liver. [Res. Commun. Chem Pathol. Pharmacol. 51, 285-288.]

### BRIEF DESCRIPTION OF THE DRAWINGS

FIGS. 1A and 2A show the influence of high copper levels in the culture on Recombinant Protein Expression. In both figures, the Y-axis represents normalized data on Recombinant Protein Titer obtained. The dashed line represents data obtained using medium with no additional copper added, i.e. only a basal level of 0.087 micromolar copper naturally present in the media. The X-axis represents bioreactor days. The solid line represents the protein titer obtained when additional copper is added.

FIGS. 1B and 2B show the influence of high copper levels on recombinant protein specific productivity. In both figures, the Y-axis represents normalized data on Recombinant Protein Specific Productivity versus bioreactor days on the X-axis. The dashed line again represents data obtained using medium with no additional copper added, i.e. only a basal level of 0.087 micromolar copper naturally present in the media. The solid line represents the protein specific productivity obtained when additional copper is added.

FIGS. 3A and 3B show Recombinant Protein Titer and Recombinant Protein Specific Productivity, respectively, versus bioreactor days for the basal level of copper found in the medium and for various levels of copper added (0.315, 0.629 and 1.259 micromolar).

FIG. 4 is a surface plot of normalized Specific Productivity (qp) vs. osmolality and copper concentration.

### DETAILED DESCRIPTION

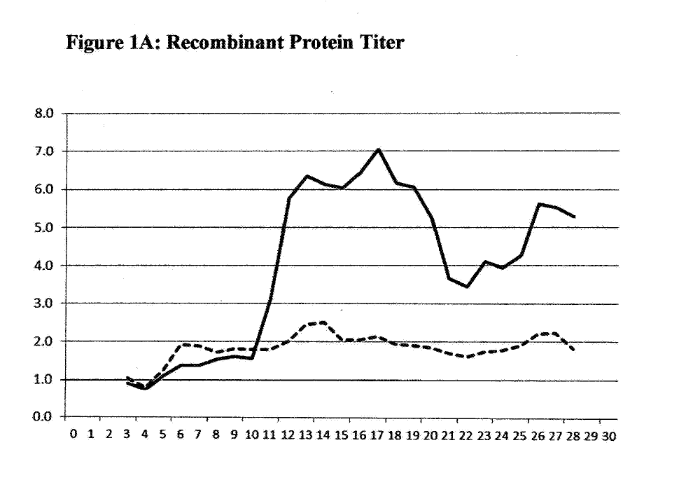

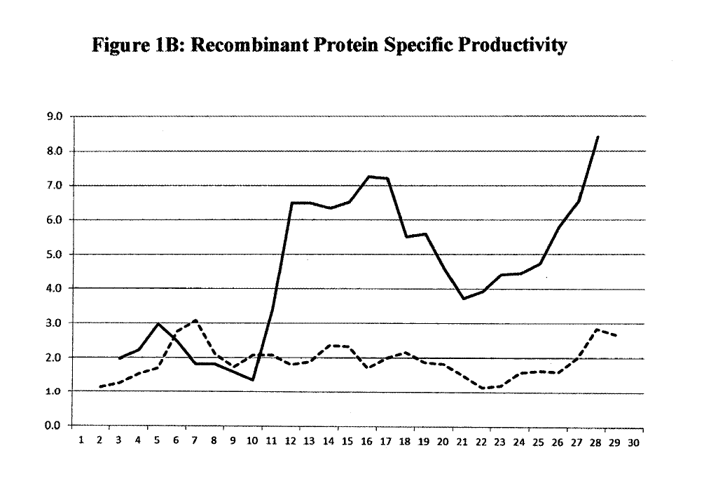

This data was generated in 2013 when the process was operated using an external membrane-based cell retention device, using medium without copper supplementation. Baseline cultures represented as (-) Copper were executed with copper levels found in normal medium in 16-160 nanomolar range. The first experimental evidence of the added benefits of copper were obtained when two (2) bioreactors received medium with copper supplemented. The addition of copper occurred on day ten (10) and showed an immediate influence on recombinant protein expression as evidenced in the graph showing the dramatic increase in protein expression. However, the cupric ion source, such as cupric sulfate or cupric chloride or other cupric salt with similar characteristics, may be added to the medium prior to adding the cells with similar results. FIG. 1 shows the influence of adding 40.9 micromolar copper to the culture medium. A four (4) to five (5) fold increase in protein expression was demonstrated through duplicate bioreactors operating at the same conditions as the baseline runs. The addition of about 40 micromolar copper in the form of cupric ion appears to give optimal results, but other additional concentrations within the range of 0.5 micromolar to about 10.0 micromolar appear to give similar results.

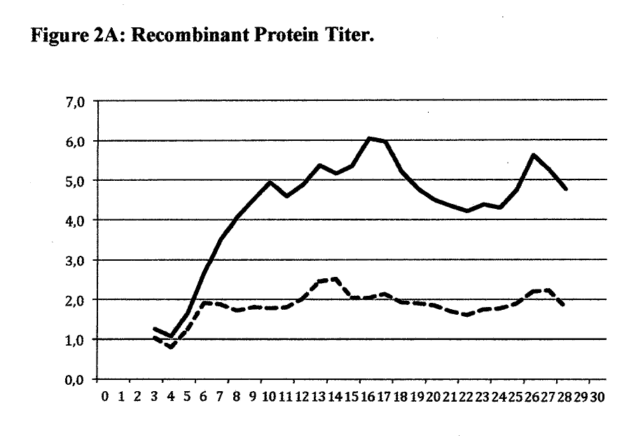

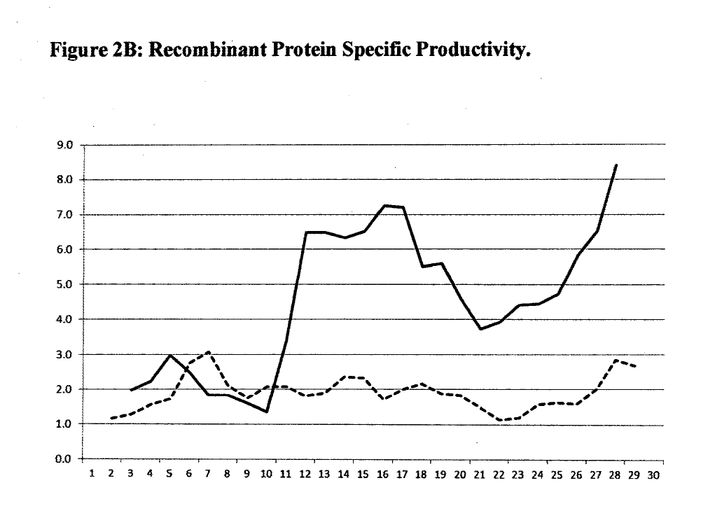

To better understand the influence of high levels of copper during the initial experimental runs, additional runs were executed using a reduced quantity of copper. FIG. 2 represents data generated using a copper addition of 7.87 micromolar. This data demonstrates that with all other factors equal to baseline bioreactors, the addition of 7.87 micromolar resulted in a three (3) to four (4) fold increase in protein expression.

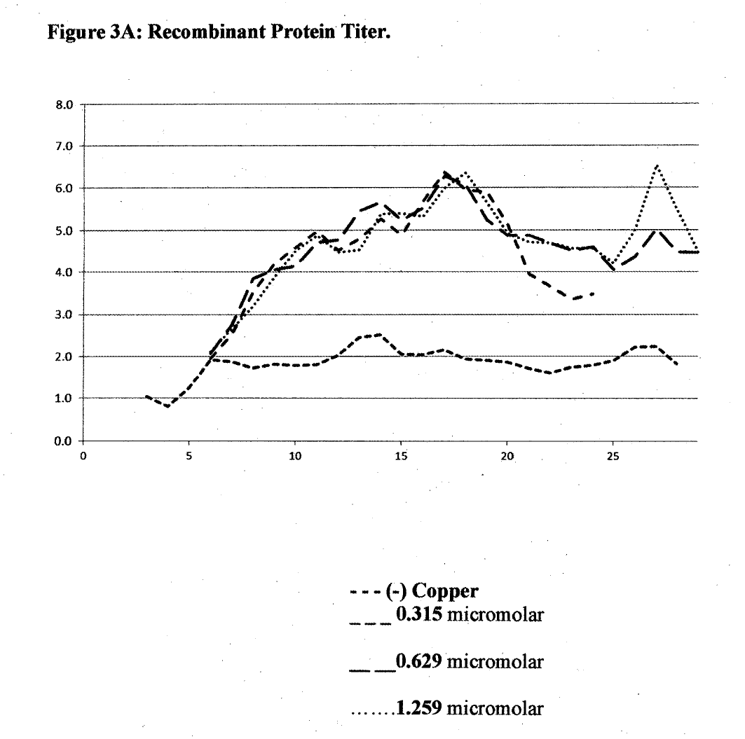

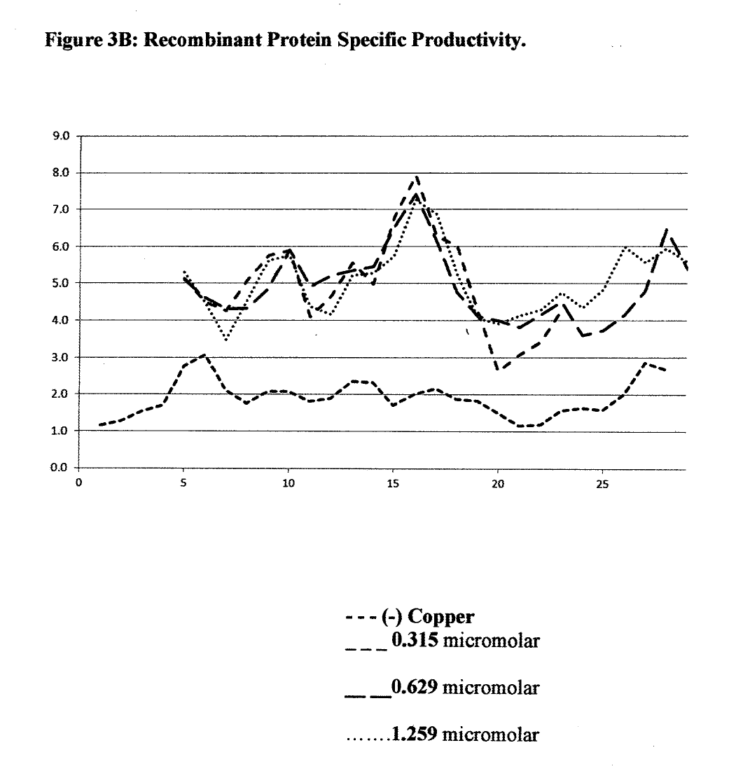

Further bioreactor experimentation was carried out to demonstrate the influence of more reasonable copper levels on protein expression. FIG. 3 represents data generated through duplicate bioreactors operated at varying levels of copper concentration through the course of the bioreactor run. All other parameters were maintained equivalent to the baseline runs. This data demonstrates when compared to the 7.87 micromolar copper addition as detailed in FIG. 2, that copper concentrations of 0.315, 0.63 and 1.26 micromolar will result in three (3) to four (4) fold increases equivalent to 7.87 micromolar.

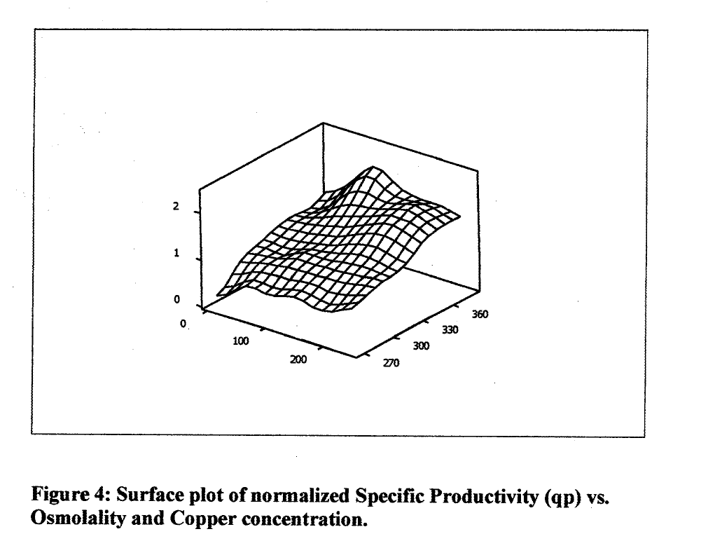

FIG. 4 shows the specific productivity on the Z (vertical) axis with the copper concentration and osmolality on the X and Y-axis respectively. This data was generated using a six day, 250 mL shake flask, batch cell culture model to determine/demonstrate the effect of added copper. The specific productivity may also be increased with increased osmolality of the medium, but the greatest effect is seen with the addition of copper ion. A response surface Design of Experiment was performed where the cultures were seeded at 0.5e6 cells/mL into basal medium supplemented with cupric chloride and or, optionally, sodium chloride to adjust the copper levels to between 0.087 to 3.78 micrmolar and osmolality to between 270 to 380 mOsmo respectively. Five different levels of each factor were chosen (0.087, 0.787, 1.495, 2.927, and 3.78 micromolar copper and 270, 310, 350, 360, 380 mOsmo). Cultures were then sampled daily for viable cell concentration determination for six days. Product concentration evaluation was performed on days 4-6. The specific productivity represents the average specific productivity between days 4 and 6 of the batch culture normalized to average specific productivity of the center point in the study (310 mOsmo, 1.49 micromolar Cu). As seen in FIG. 4 there is a clear increase in specific productivity with both increases in osmolality and increases in copper concentration. From a statistical analysis of the data from the response surface design experiment, both Cu and osmolality exhibited a highly significant effect, P=0.000 (where any P<0.05 is considered significant), on specific productivity, but there was also a statistically significant interaction between the two P=0.003, see Table 1.

Per the equation developed to model this data, the specific productivity increased from 0.134 to 0.355 with an increase in copper concentration from 0.087 to 3.78 micromolar at an osmolality of 270 and from 1.2 to 2.15 at an osmolality of 380. Similarly there is a clear increase in specific productivity from 0.143 to 1.22 with an increase osmolality from 270 to 380 at 0.087 micromolar copper and from 0.355 to 2.158 at 3.78 micromolar copper.

TABLE 1

| Term | Coef SE | Coef | T | P |
|---|---|---|---|---|
| Constant | 1.28562 | 0.03053 | 42.107 | 0.000 |
| Osmo | 0.71634 | 0.03372 | 21.245 | 0.000 |
| Cu ppb | 0.28843 | 0.03492 | 8.260 | 0.000 |
| Osmo*Osmo | 0.10210 | 0.04882 | 2.091 | 0.063 |
| Cu ppb*Cu ppb | −0.31375 | 0.05114 | −6.135 | 0.0000 |
| Osmo*Cu ppb | 0.18223 | 0.04553 | 4.002 | 0.003 |

Table one gives the coefficients for the regression model equation which fits the specific productivity data collected as a function of osmolality and copper concentration. The equation consists of a constant, two linear terms (Osmo, Cu ppb), and three nonlinear terms (Osmo*Osmo, Cu ppb*Cu ppb, Osmo*Cu ppb) as shown in the first column in table 1. The “Osmo” term represents the osmolality of the culture where as the “Cu ppb” term represents the copper concentration. The coefficients for each term are listed in the second row (Coef) with the standard error of those coefficients listed in the third row (SE Coef). The forth row is the T statistic of the coefficients and is the quotient of the Coefficient divided by the standard error of the coefficient. The larger the magnitude of the T value the larger the significance of the coefficient. The fifth column represents the p-value for each term and a value of less than 0.05 is considered to indicate statistical significance. As can be seen in table 1 all but the Osmo*Osmo term have a p-value less than 0.05 and are therefore considered significant. The final regression equation is shown below.

Qp=1.28562+0.71634*Osmo+0.28843*Cu ppb+0.10210*Osmo*Osmo−3.1375*Cu ppb*Cu ppb+0.18223*Osmo*Cu ppb

### SUMMARY

A method of increasing cell expression of mammalian cells, comprising the use of copper additives to the cell culture medium is provided herein. From about 0.5 micromolar to about 10.0 micromolar copper is preferably added to the cell culture medium. A similar addition of 0.5 micromolar copper to about 10.0 micromolar copper provides an increased cell specific productivity. Cupric ion is particularly preferred as the copper additive. The manufacturing system is composed of the augmented cell culture medium and mammalian cells. Preferred mammalian cells for use in the cell culture medium are CHO, BHK or human mammalian cells. Unstable recombinant proteins are particularly good candidates for expression utilizing a membrane-based cell retention system with copper additives. This system is useful with perfusion cell cultures to produce coagulation proteins, chosen from the group consisting of recombinant Factor VIII, B Domain Deleted recombinant Factor VIII, recombinant Factor IX and rFVII or rFVIIa.

The addition of other bulk ions such as sodium and potassium that increase the osmolality of the medium further enhance protein expression.

The method is preferably used in combination with a membrane-based cell retention system and perfusion cell culture.

Most preferred is the use of this improved method of recombinant protein expression applied to increasing the expression of B-Domain Deleted recombinant FVIII in mammalian cells with the addition of about 0.5 to about 10.0 micromolar cupric ion to the cell culture medium used with a manufacturing system, composed of perfusion cell culture used in combination with an external membrane-based cell retention system.

What is claimed is:

1. A method of increasing protein expression of mammalian cells with the addition of from about 0.5 micromolar to about 10.0 micromolar copper to the cell culture medium.

2. A method of increasing cell specific productivity with the addition of from about 0.5 micromolar to about 10.0 micromolar copper to the cell culture medium.

3. The method of claim 1, wherein the manufacturing system comprising the augmented cell culture medium and mammalian cells, is used to produce recombinant proteins.

4. The method of claim 2, wherein the manufacturing system comprising the augmented cell culture medium and mammalian cells, is used to produce recombinant proteins.

5. The method of claim 3, wherein the recombinant proteins are coagulation proteins.

6. The method of claim 3 wherein the coagulation proteins are chosen from the group consisting of recombinant Factor VIII, B Domain Deleted recombinant Factor VIII, and recombinant Factor VII or recombinant Factor VIIa.

7. The method of claim 1 wherein the mammalian cells are chosen from CHO, BHK or human mammalian cells.

8. The method of claim 1, wherein the copper is added with other bulk ions such as sodium and potassium that increase the osmolality of the medium as a further enhancement of protein expression.

9. The method of claim 1 wherein a membrane based cell retention system is used in combination with perfusion cell culture.

10. The method of claim 1 wherein the copper added is in the form of cupric ion.

11. A method of increasing the expression of B Domain Deleted recombinant Factor VIII in mammalian cells with the addition of about 0.5 to about 10.0 micromolar cupric to the cell culture medium used with a manufacturing system, composed of perfusion cell culture used in combination with an external membrane based cell retention system.
